(19) United States
(12) Patent Application Publication
(10) Pub. No.: US 2008/0213199 A1
(43) Pub. Date: Sep. 4, 2008

(54) Amphoteric Polysaccharide Compounds Containing Aldehyde Function(S), Composition Comprising Them and Cosmetic Use Thereof

(76) Inventor: Michel Philippe, Wissous (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/660,374

(22) PCT Filed: Aug. 18, 2005
(86) PCT No.: PCT/EP05/09987

§ 371 Date: Sep. 25, 2007

Related U.S. Application Data
(60) Provisional application No. 60/612,177, filed on Sep. 23, 2004.

(30) Foreign Application Priority Data
Aug. 19, 2004 (FR) 0408998

(51) Int. Cl. A61K 8/84; C08F 251/00; A61Q 17/04; A61Q 19/00; A61Q 5/00; A61Q 1/00; A61Q 5/12; A61Q 5/02

(52) U.S. Cl. 424/59; 527/309; 424/78.02; 510/130; 510/122; 510/119

(57) ABSTRACT

The present invention relates to novel amphoteric polysaccharide compounds containing aldehyde function(s) corresponding to the formula (I): P—(O—(Y)ₚ-CAT)ₘ in which: P represents a polysaccharide chain, the said chain hearing one or more aldehyde functions and optionally one or more groups —COOV, this group possibly being obtained during the oxidation of certain carbon atoms of a saccharide unit, Y represents a linear or branched, saturated or unsaturated, optionally hydroxylated divalent C₁-C₈ divalent hydrocarbon-based group comprising at least one ether and/or amine function in the hydrocarbon-based chain, or a group —Si(R)₂—[O—Si(R)₂]_{q}-Z-; p is O or 1, and CAT represents an ammonium group or a cationic polymer chain obtained by grafting and polymerization of ethylenic monomers bearing an ammonium function. The invention also relates to their use in cosmetics and to compositions comprising them.

The present invention relates to novel amphoteric polysaccharide compounds containing aldehyde function(s), to their use in cosmetics and to compositions comprising them.

In the cosmetics field, it is especially sought to improve the cosmetic properties of keratin materials, such as the hair and the skin, and more particularly sensitized hair, i.e. hair that has become damaged or embrittled, especially due to the chemical action of atmospheric agents and/or of hair treatments such as permanent-waving, dyeing or bleaching.

With this aim, it is common practice to use complementary cosmetic agents known as conditioning agents, for example cationic polymers or silicones, which are intended mainly to repair or limit the harmful or undesirable effects induced by the various treatments or attacking factors to which hair fibres are more or less repeatedly subjected. These conditioning agents also improve the cosmetic behaviour of natural-hair.

Other conditioning agents, such as the amphoteric polysaccharides described in documents U.S. Pat. No. 4,803,071, U.S. Pat. No. 4,464,523, WO 90/03779 and FR 2 883 599 may be used in cosmetic hair compositions. However, these polysaccharides are not very efficient as regards conditioning and remanence.

The Applicant has found that, after several uses, the hair becomes laden and lacks lightness.

The Applicant has thus found, surprisingly and unexpectedly, novel amphoteric polysaccharide compounds containing aldehyde function(s), which, when used in cosmetics, make it possible to overcome the drawbacks described above and to obtain excellent cosmetic properties such as an excellent conditioning and protecting effect on the hair, good disentangling of the hair, softness and a smoothing effect on keratin fibres. In addition, the use of these amphoteric polysaccharide compounds containing aldehyde function(s) leads in particular to good remanence of these properties, even after washing the hair several times, without-observing an excessive deposit that would lead to laden, non-maleable and non-supple hair.

These conditioning agents also give the skin cosmetic properties such as good moisturization.

One subject of the present invention is thus novel amphoteric polysaccharide compounds containing aldehyde function(s) as described below.

A second subject of the invention consists of a use of such a polysaccharide compound in cosmetics, as a conditioning agent, and especially for the cosmetic treatment of keratin materials, such as caring for and protecting the hair, hairstyling, permanent-waving, relaxing, dyeing or bleaching the hair, or alternatively cleansing and care of the skin, or makeup of the skin, the lips or the nails.

A subject of the invention is also a cosmetic composition comprising at least one polysaccharide compound according to the invention, in a cosmetically acceptable medium.

Other subjects, characteristics, aspects and advantages of the invention will emerge even more clearly on reading the description and the various examples that follow.

The amphoteric polysaccharide compounds containing aldehyde function(s) according to the invention bear, as substituents other than the aldehyde functions, at least one anionic group and at least one cationic group.

The amphoteric polysaccharide compounds containing aldehyde function(s) according to the invention may be represented by formula (I) below:

P—(O—(Y)ₚ-CAT)ₘ (I)

in which:
the oxygen atoms belong to one or more polysaccharide units of P,
P represents a polysaccharide chain, the said chain bearing one or more aldehyde functions (—CHO) and optionally one or more groups —COOV, this group possibly being a obtained during the oxidation of certain carbon atoms, for example in position C2, C3 or C6, of a, saccharide unit; in the case of an oxidation at C2 or C3, from 0.01% to 75% on a numerical basis and preferably from 0.1% to 50% on a numerical basis of the rings possibly having been opened;
Y represents:
   a linear or branched, saturated or unsaturated, optionally hydroxylated divalent C₁-C₈ hydrocarbon-based group optionally comprising at least one ether and/or amine function in the hydrocarbon-based chain, or
   a group

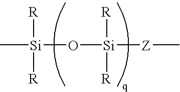

the silicon atom being attached to the oxygen atom in the group —O—(Y)ₚ-CAT,
in which each R, which may be identical or different, represents R₁ or OR₁,
R₁ represents a linear or branched, saturated or unsaturated, optionally hydroxylated C₁-C₈ and preferably C₁-C₄ monovalent hydrocarbon-based group optionally comprising at least one ether and/or amine function in the hydrocarbon-based chain,
q represents an integer ranging from 0 to 10 and preferably from 0 to 5, q=0 being particularly preferred, and
Z represents a linear or branched, saturated or unsaturated, optionally hydroxylated C₁-C₁₂ and preferably C₁-C₈, divalent hydrocarbon-based group optionally comprising at least one ether and/or amine function in the hydrocarbon-based chain,
P is equal to 0 or 1, p being 0 when CAT represents a cationic polymer chain obtained by grafting and polymerization of ethylenic monomers bearing an ammonium group,
CAT represents:
   an ammonium group

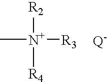

in which R₂, R₃ and R₄ represent, independently of each other, a hydrogen atom or a linear or branched, saturated or unsaturated, optionally hydroxylated C₁-C₂₂ and preferably C₁-C₁₈ monovalent hydrocarbon-based group optionally comprising at least one ether and/or amine function in the hydrocarbon-based chain, and Q′ represents a mineral or organic anion, for example a halogen atom such as a chlorine or bromine atom, the chlorine atom being particularly preferred, or an acetate, a citrate, a lactate, an oleate or a behenate, or
a cationic polymer chain obtained by grafting and polymerization of ethylenic monomers bearing an ammonium group, for example of formula:

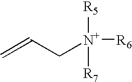

in which R₅, R₆ and R₇ represent, independently of each other, a hydrogen atom, a linear or branched C₁-C₂₂ and preferably C₁-C₁₈ alkyl group, or a linear or branched C₂-C₂₂ and preferably C₂-C₁₈ alkenyl group,
m is such that the degree of substitution of the polysaccharide compound with a cationic group (DS(+)) is within the range from 0.02 to 1.5 and preferably from 0.05 to 1.

The term “degree of substitution DS(+) of the amphoteric polysaccharide compounds” according to the invention means the ratio between the number of hydroxyl groups substituted with a cationic group for all the repeating units and the number of elementary monosaccharides (even opened by pre-oxidation) constituting the unit.

The polysaccharide chain containing aldehyde function(s), represented by P, is preferably a polysaccharide chain obtained by oxidation of a cellulose, a starch, inulin, guar gum, xanthan gum, pullulan, agar-agar, sodium, potassium or ammonium alginate, carrageenan, dextran, furcellaran, gellan gum, gum arabic, gum tragacanth, hyaluronic acid, konjac mannan, lignin sulfonate, carob gum, partially N-acetylated chitin, pectin, polydextrose, rhamsan gum or welan gum.

The oxidation may be performed according to a process known in the art, for example according to the process described in FR 2 842 200 or in the article “Hydrophobic films from maize bran hemicelluloses” by E. Fredon et al. Carbohydrate Polymers 49, 2002, pages 1 to 12.

More preferably, the polysaccharide chain containing aldehyde function(s) is obtained by oxidation of cellulose, carboxymethylcellulose, hydroxyethylcellulose, hydroxypropylcellulose, hydroxypropylmethylcellulose, methylcellulose, starch, starch acetate, hydroxyethyl starch, hydroxypropyl starch, inulin, guar gum, carboxymethylguar gum, carboxymethylhydroxypropylguar gum, hydroxyethylguar gum, hydroxypropylguar gum or xanthan gum.

The polysaccharide chain containing aldehyde function(s) preferably has a weight-average molecular mass between 500 and 15 000 000 and better still between 1000 and 10 000 000.

Examples of linear or branched, saturated or unsaturated C₁-C₈ and preferably C₁-C₆ divalent hydrocarbon-based groups Y or Z that may especially be mentioned include linear or branched C₁-C₆ alkylene groups, such as methylene, ethylene, n-propylene, isopropylene, n-butylene, tert-butylene or hexylene; linear or branched C₂-C₈ alkylene groups, such as vinylene, allylene, crotonylene, butenylene, isobutenylene, tert-butenylene, hexenylene or octenylene. These groups may also bear at least one hydroxyl substituent and/or may comprise at least one ether and/or amine function in the alkylene or alkenylene chain.

Examples of linear or branched, saturated or unsaturated C₁-C₈ and preferably C₁-C₄ monovalent hydrocarbon-based groups R₁ that may especially be mentioned include linear or branched C₁-C₄ alkyl groups, such as methyl, ethyl, n-propyl, isopropyl, n-butyl or tert-butyl; linear or branched C₂-C₄ alkenyl groups, such as vinyl, allyl, crotonyl, butenyl, isobutenyl or tert-butenyl; the said groups possibly bearing at least one hydroxyl substituent and/or possibly comprising at least one ether and/or amine function.

Examples of R₂ to R₇ that may especially be mentioned include linear or branched C₁-C₄ alkyl groups, such as methyl, ethyl, n-propyl, isopropyl, n-butyl or tert-butyl groups, and linear or branched C₁₂-C₁₈ alkyl groups, such as lauryl, myristyl, cetyl or stearyl groups. Examples of linear or branched C₂-C₁₈ and preferably C₂-C₆ alkenyl groups that may especially be mentioned include vinyl, allyl, crotonyl and butenyl groups.

The amphoteric polysaccharide compounds that are most particularly preferred in the invention are those corresponding to formula (I) in which:

P represents a polymer chain obtained by oxidation of cellulose, carboxymethylcellulose, hydroxyethylcellulose, hydroxypropylcellulose, hydroxypropylmethylcellulose, methylcellulose, starch, starch acetate, hydroxyethyl starch, hydroxypropyl starch, inulin, guar gum, carboxymethylguar gum, carboxymethylhydroxypropylguar gum, hydroxyethylguar gum, hydroxypropylguar gum or xanthan gum;
Y represents a linear or branched C₁-C₆ alkylene group, such as methylene, ethylene, n-propylene, isopropylene, n-butylene, tert-butylene or hexylene; or a linear or branched C₂-C₈ alkenylene group, such as vinylene, allylene, crotonylene, butenylene, isobutenylene, tert-butenylene, hexenylene or octenylene, or

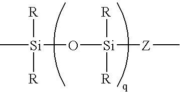

in which each R, which may be identical or different, represents R₁ or OR₁,
   R₁ represents a linear or branched C₁-C₄ alkyl group, such as methyl, ethyl, n-propyl, isopropyl, n-butyl or tert-butyl; or a linear or branched C₂-C₄ alkenyl group, such as vinyl, allyl, crotonyl, butenyl, isobutenyl or tert-butenyl;
   q represents an integer ranging from 0 to 5,
   Z represents a linear or branched C₁-C₈ alkylene group, such as methylene, ethylene, n-propylene, isopropylene, n-butylene, tert-butylene or hexylene; or a linear or branched C₂-C₈ alkenylene group, such as vinylene, allylene, crotonylene, butenylene, isobutenylene, tert-butenylene, hexenylene or octenylene,
   p, CAT and m having the same meanings as those above, and
   R₂ to R₇ represent a hydrogen atom, an optionally hydroxylated C₁₋₄ alkyl group such as methyl, ethyl, n-propyl, isopropyl, n-butyl, or tert-butyl, or an optionally hydroxylated C₁₂-C₁₈ alkyl group such as lauryl, myristyl, cetyl or stearyl, or a linear or branched C₂-C₆ alkenyl group such as vinyl, allyl, crotonyl or butenyl.

The amphoteric polysaccharide compounds containing aldehyde function(s) as described above may be used in cosmetics, as conditioning agents, especially for the cosmetic treatment of keratin materials, such as caring for and protecting the hair, hold and discipline of the hairstyle, but also for cleansing and caring for the skin and for making up the skin, the lips and the nails.

A subject of the present invention is also a cosmetic composition comprising, in a cosmetically acceptable medium, at least one amphoteric polysaccharide compound containing aldehyde function(s) according to the invention, preferably in an amount ranging from 0.05% to 50% by weight and better still from 0.5% to 25% by weight relative to the total weight of the composition.

The term “cosmetically acceptable medium” means a medium that is compatible with any keratin material, such as the skin, the hair, the nails, the eyelashes, the eyebrows and the lips and any other area of body or facial skin.

The cosmetically acceptable medium may consist solely of water or of a mixture of water and of a cosmetically acceptable solvent such as a C₁-C₄ lower alcohol, such as ethanol, isopropanol, tert-butanol or n-butanol; alkylene polyols, for instance propylene glycol; polyol ethers; and mixtures thereof.

The composition according to the invention may also comprise one or more standard additives that are well known in the art, such as anionic, cationic, nonionic, amphoteric or zwitterionic surfactants, anionic, cationic, nonionic, amphoteric or zwitterionic polymers, thickeners, nacreous agents, opacifiers, UV-screening agents, fragrances, mineral, plant and/or synthetic oils, fatty acid esters, dyes, volatile or non-volatile, organomodified or non-organomodified, cyclic or acyclic, branched or unbranched silicones, mineral or organic, natural or synthetic particles, preserving agents and pH stabilizers.

A person skilled in the art will take care to select the optional additives and the amount thereof such that they do not harm the properties of the compositions of the present invention.

These additives are generally present in the composition according to the invention in an amount ranging from 0 to 20% by weight relative to the total weight of the composition.

The cosmetic compositions in accordance with the invention may be in the form of a mousse, a gel, a spray or a lacquer and may be used in rinse-out or leave-in application.

The compositions in accordance with the invention may be used as hair products, especially rinse-out or leave-in products, and in particular for washing, caring for and/or conditioning the hair, holding the hairstyle, and shaping, dyeing, bleaching, permanently reshaping or relaxing the hair.

The compositions of the invention may also be used as care or hygiene products such as protective, treating or care creams for the face, the hands or the body, protective or care body milks, gels or mousses for caring for or cleansing the skin, or alternatively as products for making up or for removing makeup from the skin, the lips, the nails and the eyelashes.

The examples below are given as illustrations of the invention.

### EXAMPLES

### Example 1

### Grafting of 3-epoxypropyltrimethylammonium chloride onto oxidized starch

24 g of oxidized starch (acid number: 1.4 mmol/g, carbonyl number 1.08 mmol/g) are dispersed in 500 ml of acetonitrile with stirring.

62.5 mmol of 50% sodium hydroxide and 0.2 mol of 3-epoxypropyltrimethylammonium chloride are successively added. The mixture is heated at 50° C. for 5 hours and then neutralized with 12 ml of acetic acid.

A brown gum is obtained, which is reslurried twice in acetonitrile: the acetonitrile penetrates the gum and allows certain impurities to be extracted, which are then removed by suction-filtering the gum, this manipulation being repeated twice.

The solid is dried under vacuum at 50° C. until a constant weight is obtained. A degree of grafting of 78% is determined by measuring the chloride number on the filtrates and the solid.

### Example 2

### Shampoo

A shampoo was prepared using the following ingredients, the amounts of which are given as weight percentages of active material relative to the total weight of the composition:

| | |
|---|---|
| Sodium lauryl ether sulfate (Texapon N702 from Cognis) | 12.5% |
| Cocoylbetaïne (Dehyton AB 30 from Goldschmidt) | 2.5% |
| Dimethicone (DC200 Fluid from Dow Corning) | 2.0% |
| Compound of Example 1 | 0.5% |
| Cocamide monoisopropanolamine | 0.4% |
| Carbomer | 0.2% |
| Preserving agent | qs |
| Fragrance | qs |
| Citric acid/sodium hydroxide qs | pH 6.5 |
| Water qs | 100 |

### Example 3

### Conditioner

A conditioner was prepared using, the following ingredients, the amounts of which are given as weight percentages of active material relative to the total weight of the composition:

| | |
|---|---|
| Behenyltrimethylammonium chloride (Genamin KDMP | 1.2% |
| from Clariant) | |
| PEG/PPG Dimethicone (Abil B8851 from Goldschmidt) | 0.5% |
| Cyclopentasiloxane (Dow Corning 245 Fluid) | 15.0% |
| Compound of Example 1 | 1.0% |
| Propylene glycol | 2.5% |
| Preserving agent | qs |
| Fragrance | qs |
| Citric acid/sodium hydroxide qs | pH 6.5 |
| Water qs | 100 |

### Examples 4-6

Dye compositions were prepared using the following ingredients, the amounts of which are given as weight percentages of active material relative to the total weight of the composition:

| | Ex. 4 | Ex. 5 | Ex. 6 |
|---|---|---|---|
| para-Phenylenediamine | 0.24 | 0.24 | 0.24 |
| para-Aminophenol | 0.44 | 0.44 | 0.44 |
| 2-Aminophenol | 0.028 | 0.028 | 0.028 |
| 1,3-Dihydroxybenzene | 0.192 | 0.192 | 0.192 |
| 3-Aminophenol | 0.019 | 0.019 | 0.019 |
| 5-N-(β-Hydroxyethyl)amino-2-methylphenol | 0.021 | 0.021 | 0.021 |
| 1,3-Dihydroxy-2-methylbenzene | 0.055 | 0.055 | 0.055 |
| Anhydrous sodium metasilicate | 2 | 2 | 2 |
| Monoethanolamine | 5.45 | 5.45 | 5.45 |
| Reducing agent, antioxidant, sequestering | qs | qs | qs |
| agent, fragrance | | | |
| Propylene glycol | 10 | 10 | 10 |
| Crosslinked acrylic acid polymer | 0.4 | 0.4 | 0.4 |
| Compound of Example 1 | 1.5 | 1.5 | 2.8 |
| Cationic polymer: hexadimethrine chloride | 3 | 3 | — |
| (CTFA name) Mexomer PO sold by the | | | |
| company Chimex | | | |
| Powdered sodium lauryl sulfate | 3 | — | — |
| Lauryl alcohol oxyethylenated with 12 mol | — | 7.5 | 7.5 |
| of ethylene oxide | | | |
| Oleocetyl alcohol oxyethylenated with | — | 4 | 4 |
| 30 mol of ethylene oxide | | | |
| Decyl alcohol oxyethylenated with 3 mol of | 10 | 10 | 10 |
| ethylene oxide | | | |
| Decyl alcohol oxyethylenated with 5 mol of | 8 | — | — |
| ethylene oxide | | | |
| Lauric acid | 2.5 | 2.5 | 2.5 |
| 50/50 cetylstearyl alcohol | 11.5 | 11.5 | 11.5 |
| Nacreous agent: hydrophobic fumed silica | 1.2 | 1.2 | 1.2 |
| Nacreous agent: glyceryl monostearate | 2 | 2 | 2 |
| Demineralized water qs | 100 | 100 | 100 |

At the time of use, each dye composition described above was mixed weight-for-weight with a 20-volumes hydrogen peroxide solution (6% by weight).

The mixtures thus prepared were applied for 30 minutes to locks of natural or permanent-waved grey hair containing 90% white hairs. The locks were then rinsed, washed with a standard shampoo, rinsed again and then dried.

The hair was dyed in a golden-blond shade for each of the Examples 4 to 6.

### Example 7

Another dye composition was prepared using the following ingredients, the amounts of which are given as weight percentages of active material relative to the total weight of the composition:

| | |
|---|---|
| Mixture of C18 to C24 linear alcohols | 3 |
| (C18/C20/C22/C24: 7/57/30/6 - alcohol content >95%) | |
| Oxyethylenated stearyl alcohol (2 mol of ethylene oxide) | 4.5 |
| Oxyethylenated stearyl alcohol (21 mol of ethylene oxide) | 1.75 |
| Oleic acid | 2.6 |
| Cationic polyurethane obtained by condensation of 1,3-bis- | 0.2 |
| (isocyanatomethylcyclohexane), N,N-dimethylethanolamine | |
| quaternized with bromododecane, N,N-dimethylethanolamine | |
| and polyoxyethylene of molecular weight 10 000 | |
| Crosslinked poly(acrylic acid) (product sold under the name | 0.4 |
| Carbopol 980 by the company Noveon) | |
| Hydroxypropylmethylcellulose | 0.2 |
| Coconut acid monoisopropanolamide | 3 |
| Merquat 100 as an aqueous 40% solution | 1.6 |
| Compound of Example 1 | 2 |
| Sodium metabisulfite | 0.71 |
| EDTA (ethylenediaminetetraacetic acid) | 0.2 |
| tert-Butylhydroquinone | 0.3 |
| 1,4-Diaminobenzene | 0.2 |
| para-Aminophenol | 1.2 |
| 1,3-Dihydroxybenzene | 0.1 |
| 1-Hydroxy-3-aminobenzene | 0.2 |
| 1-Methyl-2-hydroxy-4-β-hydroxyethylaminobenzene | 0.8 |
| Monoethanolamine | 1 |
| Aqueous ammonia containing 20% NH₃ | 11 |
| Fragrance | qs |
| Demineralized water qs | 100 |

This composition is mixed at the time of use with an oxidizing composition in emulsion form containing as oxidizing agent 7.5% hydrogen peroxide, in a proportion of 1 part by weight of dye composition per 1.5 parts by weight of oxidizing composition. The mixture obtained is applied to locks of natural hair containing 90% white hairs, and is left to act for 30 minutes. After rinsing, washing with shampoo and drying, hair dyed in a strong coppery-red light-chestnut shade is obtained.

### Example 8

The following compositions were prepared, the percentages indicated being weight percentages relative to the total weight of the composition:

### Oxidizing Composition:

| | |
|---|---|
| Fatty alcohol | 2.3% |
| Oxyethylenated fatty alcohol | 0.6% |
| Fatty amide | 0.9% |
| Glycerol | 0.5% |
| Hydrogen peroxide | 7.5% |
| Fragrance | qs |
| Demineralized water qs | 100% |

### Dye Composition:

| | |
|---|---|
| Mixture of C18 to C24 linear alcohols | 3% |
| [C18/C20/C22/C24, 7/58/30/6, alcohol content | |
| >95%] (Nafol 20-22) | |
| Mixture of C18 to C24 linear alcohols | 1.35% |
| [C18/C20/C22/C24, 7/58/30/6, alcohol content | |
| >95%] in oxyethylenated form (30 mol of ethylene | |
| oxide) (Nafolox 20-22) | |
| Oxyethylenated stearyl alcohol (2 mol of ethylene | 4% |
| oxide) | |
| Oxyethylenated stearyl alcohol (21 mol of ethylene | 2% |
| oxide) | |
| Oleic acid | 2.6% |
| Glycol distearate | 2% |
| Propylene glycol | 5% |
| Coconut acid monoisopropanolamide | 2% |
| Aculyn 44 sold by the company Rohm & Haas | 1.4% AM* |
| Crosslinked poly(acrylic acid) | 0.6% |
| Compound of Example 1 | 3% AM* |
| Merquat 100 sold by the company Calgon | 0.4% AM* |
| Reducing agents | 0.7% |
| Sequestering agents | 0.2% |
| 1,3-Dihydroxybenzene (resorcinol) | 0.6% |
| 1,4-Diaminobenzene | 0.5% |
| 1-Hydroxy-3-aminobenzene | 0.1% |
| 1-Hydroxy-2-aminobenzene | 0.05% |
| 1-Hydroxy-4-aminobenzene | 0.09% |
| 6-Hydroxybenzomorpholine | 0.017% |
| 1-β-Hydroxyethyloxy-2,4-diaminobenzene | 0.039% |
| dihydrochloride | |
| Propylene glycol monobutyl ether | 2.5% |
| Pure monoethanolamine | 1.06% |
| Aqueous ammonia (containing 20.5% ammonia) | 11.1% |
| Water qs | 100% |

AM* = Active Material

The dye composition was mixed, at the time of use, in a plastic bowl and for 2 minutes, with the oxidizing composition given above, in a proportion of 1 part of dye composition per 1.5 parts of oxidizing composition.
The mixture obtained was applied to locks of natural hair containing 90% white hairs, and was left to act for 30 minutes.
The locks were then rinsed with water, washed with shampoo, rinsed again with water and then dried and disentangled.

The hair was then dyed in a strong light-chestnut shade.

### Example 9

Another dye composition was prepared using the following ingredients, the amounts of which are given as weight percentages of active material relative to the total weight of the composition:

| | |
|---|---|
| Oleocetyl alcohol oxyethylenated with 30 mol of ethylene | 7% |
| oxide (nCA = 17-HLB = 16.5) | |
| Lauryl alcohol (C12-C14/55-45%) oxyethylenated with | 8% |
| 12 mol of ethlene oxide (nCA = 12.5-HLB = 14) | |
| Cetylstearyl alcohol (C16/C18-50/50)(nCB = 17-HLB = 1) | 5% |
| Decyl alcohol (C10-C12-C14/85-8.5-6.5) oxyethylenated | 22% |
| with 3.5 mol of ethylene oxide, sold under the name | |
| Mergital BL 309 by the company Henkel (nCB = 10.4- | |
| HLB = 8.5) | |
| Copolymer of diallyldimethylammonium chloride and of | 3% AM |
| acrylic acid, sold under the name Merquat 280 by the | |
| company Calgon, containing 35% AM | |
| Compound of Example 1 | 1% |
| Crosslinked poly(acrylic acid) sold under the name | 0.4% |
| Carbopol 934 (MW 3 000 000) by the company Goodrich | |
| Propylene glycol | 8% |
| Monoethanolamine | 8.3% |
| Hydroquinone | 0.1% |
| 1-Phenyl-3-methyl-5-pyrazolone | 0.1% |
| Aqueous sodium bisulfite solution containing 35% AM | 1.3% |
| para-Phenylenediamine | 0.5% |
| m-Dihydroxybenezene | 0.4% |
| Fragrance, sequestering agent | qs |
| Eau qs | 100% |
| pH = 11.0 | |

AM: Active Material

1. Amphoteric polysaccharide compound containing aldehyde function(s) corresponding to formula (I):
P—(O—(Y)ₚ-CAT)ₘ (I)
in which:
the oxygen atoms belong to one or more polysaccharide units of P,
P represents a polysaccharide chain, the said chain bearing one or more aldehyde functions and optionally one or more groups —COOV, this group possibly being obtained during the oxidation of certain carbon atoms of a saccharide unit,
Y represents:
a linear or branched, saturated or unsaturated, optionally hydroxylated divalent C₁-C₈ hydrocarbon-based group optionally comprising at least one ether and/or amine function in the hydrocarbon-based chain, or
a group 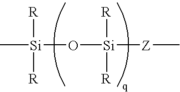
the silicon atom being attached to the oxygen atom in the group —O—(Y)ₚ-CAT,
in which each R, which may be identical or different, represents R₁ or —OR₁,
R₁ represents a linear or branched, saturated or unsaturated, optionally hydroxylated C₁-C₈ monovalent hydrocarbon-based group optionally comprising at least one ether and/or amine function in the hydrocarbon-based chain,
q represents an integer ranging from 0 to 10, and
Z represents a linear or branched, saturated or unsaturated, optionally hydroxylated C₁-C₁₂ divalent hydrocarbon-based group optionally comprising at least one ether and/or amine function in the hydrocarbon-based chain,
p is equal to 0 or 1, p being 0 when CAT represents a cationic polymer chain obtained by grafting and polymerization of ethylenic monomers bearing an ammonium group,
CAT represents:
an ammonium group 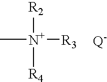
in which R₂, R₃ and R₄ represent, independently of each other, a hydrogen atom or a linear or branched, saturated or unsaturated, optionally hydroxylated C₁-C₂₂ monovalent hydrocarbon-based group optionally comprising at least one ether and/or amine function in the hydrocarbon-based chain, and Q represents a mineral or organic anion, or
a cationic polymer chain obtained by grafting and polymerization of ethylenic monomers bearing an ammonium group,
m is such that the degree of substitution of the polysaccharide compound with a cationic group is within the range from 0.02 to 1.5.

2. Amphoteric polysaccharide compound according to claim 1, characterized in that P represents a polysaccharide chain containing aldehyde function(s) obtained by oxidation of a cellulose, a starch, inulin, guar gum, xanthan gum, pullulan, agar-agar, sodium, potassium or ammonium alginate, carrageenan, dextran, furcellaran, gellan gum, gum arabic, gum tragacanth, hyaluronic acid, konjac mannan, lignin sulfonate, carob gum, partially N-acetylated chitin, pectin, polydextrose, rhamsan gum or welan gum.

3. Amphoteric polysaccharide compound according to claim 2, characterized in that P represents a polysaccharide chain containing aldehyde function(s) obtained by oxidation of a cellulose, carboxymethylcellulose, hydroxyethylcellulose, hydroxypropylcellulose, hydroxypropylmethylcellulose, methylcellulose, starch, starch acetate, hydroxyethyl starch, hydroxypropyl starch, inulin, guar gum, carboxymethylguar gum, carboxymethylhydroxypropylguar gum, hydroxyethylguar gum; hydroxypropylguar gum or xanthan gum.

4. Amphoteric polysaccharide compound according to any one of the preceding claims, characterized in that the divalent hydrocarbon-based group Y or Z is chosen from linear or branched C₁-C₆ alkylene groups and linear or branched C₂-C₈ alkenylene groups, optionally bearing at least one hydroxyl group and/or optionally comprising at least one ether and/or amine function in the alkylene or alkenylene chain.

5. Amphoteric polysaccharide compound according to claim 4, characterized in that the divalent hydrocarbon-based group Y or Z is chosen from methylene, ethylene, n-propylene, isopropylene, n-butylene, tert-butylene, hexylene, vinylene, allylene, crotonylene, butenylene, isobutenylene, tert-butenylene, hexenylene and octenylene groups.

6. Amphoteric polysaccharide compound according to any one of the preceding claims, characterized in that R₁ represents a linear or branched C₁-C₄ alkyl group or a linear or branched C₂-C₄ alkenyl group.

7. Amphoteric polysaccharide compound according to claim 6, characterized in that R₁ is chosen from methyl, ethyl, n-propyl, isopropyl, n-butyl, tert-butyl, vinyl, allyl, crotonyl, butenyl, isobutenyl and tert-butenyl groups.

8. Amphoteric polysaccharide compound according to any one of the preceding claims, characterized in that Q represents a halogen atom, an acetate, a citrate, a lactate, an oleate or a behenate.

9. Amphoteric polysaccharide compound according to any one of the preceding claims, characterized in that R₂, R₃ and R₄ represent, independently of each other, a hydrogen atom, a linear or branched C₁-C₄ alkyl group, a linear or branched C₁₂-C₁₈ alkyl group or a linear or branched C₂-C₆ alkenyl group.

10. Amphoteric polysaccharide compound according to claim 9, characterized in that R₂, R₃ and R₄ represent, independently of each other, a hydrogen atom or a methyl, ethyl, n-propyl, isopropyl, n-butyl, tert-butyl, lauryl, myristyl, cetyl, stearyl, vinyl, allyl, crotonyl or butenyl group.

11. Amphoteric polysaccharide compound according to any one of the preceding claims, characterized in that the ethylenic monomers, bearing a quaternary ammonium group correspond to the formula: 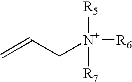
in which R₅, R₆ and R₇ represents, independently of each other, a hydrogen atom, a linear or branched C₁-C₂₂ alkyl group or a linear or branched C₂-C₂₂ alkenyl group.

12. Amphoteric polysaccharide compound according to claim 11, characterized in that R₅, R₆ and R₇ represent, independently of each other, a hydrogen atom, a linear or branched C₁-C₄ alkyl group, a linear or branched C₁₂-C₁₈ alkyl group or a linear or branched C₂-C₆ alkenyl group.

13. Amphoteric polysaccharide compound according to claim 12, characterized in that R₅, R₆ and R₇ represent, independently of each other, a hydrogen atom or a methyl, ethyl, n-propyl, isopropyl, n-butyl, tert-butyl, lauryl, myristyl, cetyl, stearyl, vinyl, allyl, crotonyl or butenyl group.

14. Use of at least one amphoteric polysaccharide compound containing aldehyde function(s) according to any one of the preceding claims, in cosmetics, as a conditioning agent.

15. Use according to claim 14, for the cosmetic treatment of keratin materials.

16. Use according to claim 15, for caring for and protecting the hair.

17. Use according to claim 15, for cleansing and caring for the skin.

18. Use according to claim 15, for making up the skin, the lips and the nails.

19. Cosmetic composition comprising, in a cosmetically acceptable medium, at least one amphoteric polysaccharide compound containing aldehyde function(s) according to any one of claims 1 to 13.

20. Cosmetic composition according to claim 19, characterized in that it comprises the said amphoteric polysaccharide compound containing aldehyde function(s) in an amount ranging from 0.05% to 50% by weight relative to the total weight of the composition.

21. Cosmetic composition according to claim 20, characterized in that it comprises the said amphoteric polysaccharide compound containing aldehyde function(s) in an amount-ranging from 0.5% to 25% by weight relative to the total weight of the composition.

22. Cosmetic composition according to any one of claims 19 to 21, characterized in that the cosmetically acceptable medium comprises water or a mixture of water and of at least one organic solvent.

23. Cosmetic composition according to claim 22, characterized in that the organic solvent is chosen from C₁-C₄ lower alcohols, alkylene polyols, polyol ethers, and mixtures thereof.

24. Cosmetic composition according to any one of claims 19 to 23, characterized in that it comprises at least one additive chosen from anionic, cationic, nonionic, amphoteric or zwitterionic surfactants, anionic, cationic, nonionic, amphoteric or zwitterionic polymers, thickeners, nacreous agents, opacifiers, UV-screening agents, fragrances, mineral, plant and/or synthetic oils, fatty acid esters, dyes, volatile or non-volatile, organomodified or non-organomodified, cyclic or acyclic, branched or unbranched silicones, mineral or organic, natural or synthetic particles, preserving agents and pH stabilizers.
